(19) United States
(12) Patent Application Publication
(10) Pub. No.: US 2016/0374828 A1
(43) Pub. Date: Dec. 29, 2016

(54) MODULAR INTERBODY ALIGNMENT DEVICE

(71) Applicant: STERLING THERAPEUTICS, LLC, Colts Neck, NJ (US)

(72) Inventor: GORDON D. DONALD, Oceanport, NJ (US)

(21) Appl. No.: 15/191,196

(22) Filed: Jun. 23, 2016

Related U.S. Application Data
(60) Provisional application No. 62/183,426, filed on Jun. 23, 2015.

(51) Int. Cl. A61F 2/44; A61F 2/46; A61B 17/02

(52) U.S. Cl. CPC A61F 2/4455; A61B 17/025; A61F 2/4611; A61B 2017/0256

(57) ABSTRACT

A modular implant device for placement between two adjacent vertebrae during a surgical procedure. The implant device comprises a distractor/nosecone portion configured for placement in a first location between the vertebrae and providing distraction and fixation of a desired vertical separation between the vertebrae at the first location. An alignment guide being a long slender element has a first end inserted into the distractor/nosecone portion and extending away from the distractor/nosecone portion, where the alignment guide is used to position the distractor/nosecone portion in the first location between the vertebrae. A body portion is configured for placement in a second location between the vertebrae, providing distraction and fixation of a desired vertical separation between the vertebrae at the second location, where the body portion includes a bore to receive the alignment guide, and the body portion is translated along the alignment guide into position between the vertebrae.

### CROSS-REFERENCE TO RELATED APPLICATIONS

This application claims the benefit of the priority date of U.S. Provisional Patent Application Ser. No. 62/183,426, titled, Modular Interbody Alignment Device, filed Jun. 23, 2015.

### BACKGROUND OF THE INVENTION

### Field of the Invention

The invention relates to the field of interbody devices, and more specifically, to a modular interbody device and alignment guide for use during a surgical implant procedure, particularly a spinal fusion procedure.

### Description of the Related Art

Implants are typically placed as unitary (single piece) devices that require space distraction (separation of bones) and proper device placement simultaneously. Placement of a large unitary device is much more difficult than a small component. Guidewires have been described and used for placement of implants but such wires are not fixated and provide no structural advantage while implanting the device/implant.

### SUMMARY OF THE INVENTION

In one or more embodiments, a modular interbody alignment device is disclosed having a distractor portion and an alignment guide extending therefrom. A subsequent implant or implants, or parts thereof may be coupled to the distractor portion using the alignment guide.

In accordance with one embodiment, the distractor portion (also referred to herein as a nosecone) with the alignment guide coupled thereto is employed to provide initial space distraction, such as intervertebral space, reconstitution of disc space height and proper device placement. Once the distractor portion/nosecone is properly positioned, the alignment guide extending therefrom is, by virtue of the proper position of the nosecone, properly positioned for receipt of the subsequent implant part(s).

The subsequent implant part may be a principal portion of an implant, and may provide the bulk of interbody structural support. The subsequent implant may be referred to herein as the “body portion” of the implant. The alignment guide may serve as fixation component operable to fix the distractor portion/nosecone and body portion one to the other.

In another embodiment, an interbody device includes an implant having a first, distractor portion and a second, separate body portion which may be coupled to the distractor portion. The distractor portion may include an opening for receiving an alignment guide, or may include an integral alignment guide extending therefrom. The body portion includes a bore for receiving the alignment guide. The bore facilitates slidable engagement between the body portion and the alignment guide. The alignment guide may be configured and operable to not only bring the distractor portion and body portion into contact, but also to fix them together. Alternatively, or in addition, the distractor portion and the body portion may have complementary mating surfaces operable to connect the portions.

The alignment guide may be “keyed” to allow for proper placement of subsequent implants without malrotation.

Devices in accordance with one or more embodiments may be employed in various surgical applications, including but not limited to ALIF (Anterior Lumbar Interbody Fusion), PLIF (Posterior), TLIF (Transforaminal) and LLIF (Lateral) interspinous fixation, fracture fixation, endosurgery, cardiovascular surgery, etc.

For example, in the case of an interbody implant, a first portion of an implant, or nosecone, acts as an interbody distractor and trial. This results in easier placement of the second portion, or body portion, of the implant with lessened risk of endplate damage. The distractor/nosecone portion is part of the final implant system. The small footprint of the nosecone portion provides for reliable and correct positioning of the whole implant. The first portion/nosecone of the implant may be stabilized by ancillary endplate fixation. The alignment guide may be used to secure and fixate first and second implant portions.

### BRIEF DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a perspective view illustration of a device in accordance with one or more embodiments of the present disclosure;

FIG. 2 is a front view illustration of a body portion of the device of FIG. 1;

FIG. 3 is an anterior view illustration of a distractor portion and alignment guide positioned in an intervertebral space according to an embodiment of the present disclosure;

FIG. 4 is a lateral view illustration of the distractor portion and alignment guide positioned in an intervertebral space according to FIG. 3;

FIG. 5 is an anterior view illustration of a distractor portion, alignment guide and body portion of an implant positioned in an intervertebral space according to an embodiment of the present disclosure;

FIG. 6 is a lateral view illustration of the distractor portion, alignment guide and body portion of an implant positioned in an intervertebral space according to FIG. 5; and

FIG. 7 is an anterior view illustration of a distractor portion, alignment guide and body portion of an implant positioned in an intervertebral space according to an embodiment of the present disclosure.

### DETAILED DESCRIPTION OF THE EMBODIMENTS

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout. As used herein the term “and/or” includes any and all combinations of one or more of the associated listed items.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

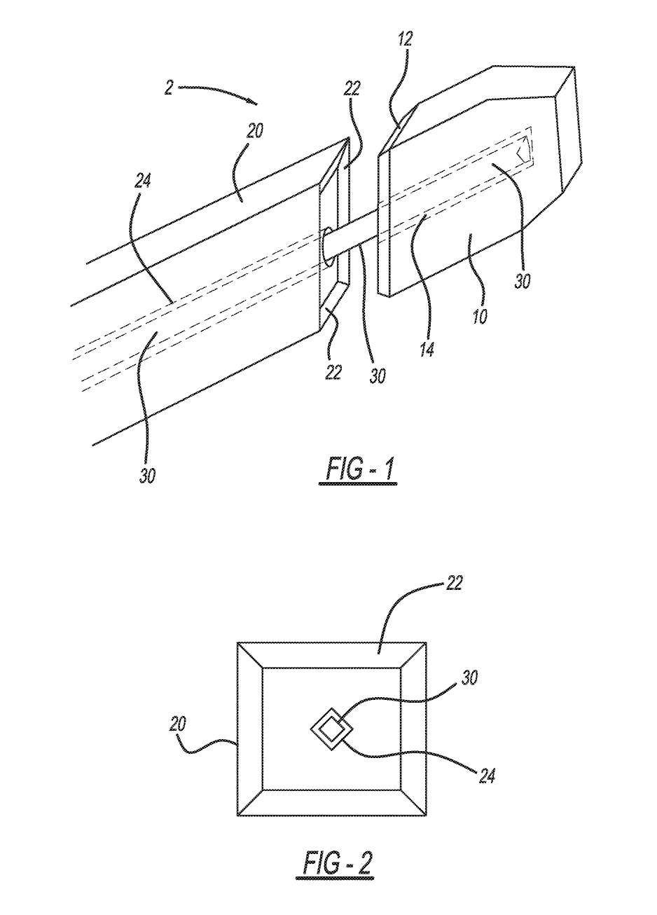

Embodiments of the present invention are described with reference to the Figures. Now referring to FIG. 1, an interbody device 2 includes a distractor portion 10, and a body portion 20 which may be coupled to each other.

The distractor portion, or nosecone, 10 may be any distractor configured similar to distractors known to those skilled in the art. For example, distractor/nosecone portion 10 may have a wedge-shaped cross section as is known in the art. However, the distractor/nosecone 10 includes an opening or bore 14 formed in an end thereof for receiving an alignment guide 30. Alternatively, the distractor/nosecone portion 10 may include an integral alignment guide 30 extending from a first end. Further, the distractor/nosecone portion 10 may include a mating element 12 configured to mate with a complementary mating element 22 of the body portion 20.

The distractor/nosecone portion 10 is operable to provide more precise placement of an implant due to its smaller size than a full implant. The alignment guide 30 extending from the distractor/nosecone 10 is easily manipulated by a surgeon to move the distractor/nosecone 10 to a desired location, such that the alignment guide 30 extending from the distractor/nosecone 10 is positioned to receive a subsequent, or body, portion 20 of an implant that can be slid along the alignment guide 30 to the desired position.

Moreover, the distractor/nosecone portion 10 is operable to perform initial interbody distraction. No separate trial or distractor is required, leaving more operating space for a surgeon. The distractor/nosecone portion 10 may be left in place as part of an implant. The distractor/nosecone portion 10 provides posterior disc space (middle column) height and restoration/maintenance of foraminal volume (the volume or space of the foramina, or openings, which are present between every adjacent pair of vertebrae).

In one embodiment, the distractor/nosecone 10 has a fixed size and shape. In other embodiments, the distractor/nosecone 10 is expandable. For example, the distractor/nosecone 10 may be expandable in vertical height to provide additional intervertebral volume and alignment. Further, it may be expandable or deploy a fixation mechanism to provide substantial anchoring or fixation to one or more vertebral endplate(s). Fixation may be achieved using screws, blades or the like. In one embodiment the expandable distractor/nosecone 10 is expandable and/or compressible independent of the other parts of the device to achieve desirable lordosis (inward curvature of the spine).

In one or more embodiments, an expandable/compressible distractor/nosecone 10 may include opposing faces having an expanding element disposed therebetween, in the manner of lifts or jacks (such as but not limited to trolley jacks or scissor jacks) as known in the art. For example, a screw type expander provides the user with the ability to expand or collapse the distractor/nosecone 10. In other embodiments a cam mechanism, or deployable spikes, may be employed to provide expansion/contraction. The distractor/nosecone portion 10 may be fixated to vertebral bodies, allowing for the body portion 20 to be mated to the distractor/nosecone portion 10 in a controlled fashion without impaction.

The body portion 20 may be any implant device, such as but not limited to an intervertebral cage, arthroplasty device, biologic or nonbiologic delivery device, etc. Regardless of the configuration of the body portion 20, it includes a bore 24 for receiving the alignment guide 30. The bore 24 is positioned and configured to permit the body portion 20 to be slidably engaged on the alignment guide 30 and brought into contact with the distractor/nosecone portion 10. The body portion 20 includes a complementary mating element 22 engageable with the mating element 12 of the distractor/nosecone portion 10. The elements 10 and 12 facilitate engagement of the body portion 20 with the distractor/nosecone portion 10. The mating elements 10 and 12 may take any suitable, known form, such as but not limited to Morse taper, mechanical interlock, magnetic connection, etc.

The alignment guide 30 is a wire, rod or the like which may be integral with or removably attachable to the distractor/nosecone portion 10. For example, the alignment guide may be threaded into the bore 14 of the distractor/nosecone portion 10. The alignment guide 30 may have a circular cross section to permit rotation in the bore 24. Alternatively, as shown in FIG. 2, the alignment guide 30 may have a non-circular cross section keyed to the cross section of the bore 24 to prevent unwanted rotation. The alignment guide 30 may be rigid or flexible, straight or curved, depending on the application, to permit better manipulation by the surgeon in the operating space.

The placement and orientation of the alignment guide 30, extending as it does from the distractor/nosecone portion 10, is dependent on placement of the distractor/nosecone portion 10. The alignment guide 30 provides for proper navigation alignment and stable positioning of the distractor/nosecone 10 and body portion 20 of the implant device 2.

The alignment guide 30 may also be used for ancillary implant construction such as but not limited to biologic or non-biologic delivery devices or implants, mechanical implants, rigid or non-rigid support devices, spacers, etc.

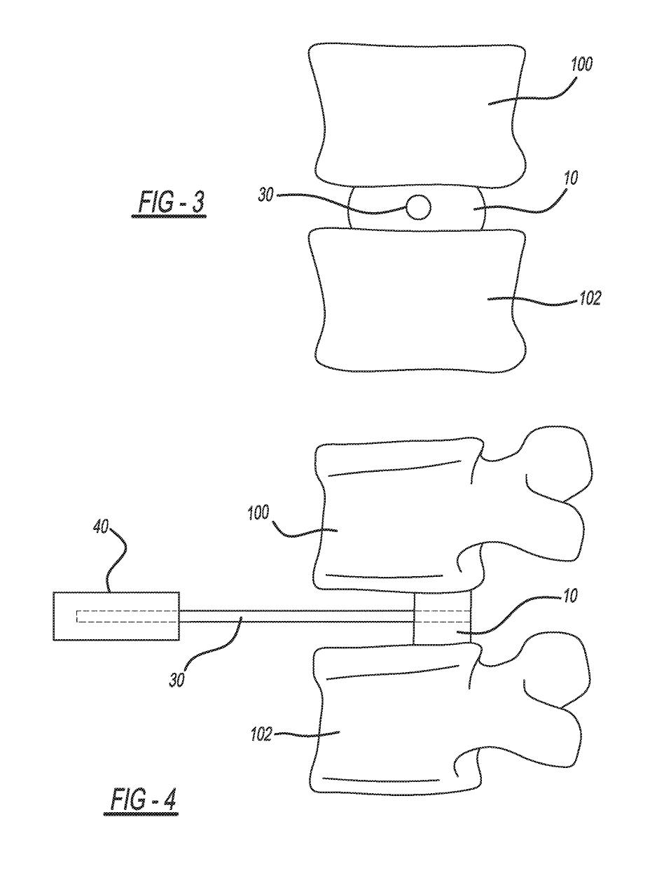

In FIGS. 3-7, a series of placement steps are shown, viewed from both the front and side of the patient, illustrating the deployment of the device 2 within the patient. These placement steps can be generally outlined as follows. After disc space preparation, the distractor/nosecone portion 10, and the alignment guide 30 are inserted and properly positioned in the interspace. The distractor/nosecone portion 10 may be manipulated by the surgeon using the alignment guide 30 to adjust the interspace. A handle 40 may be provided which is attachable to the alignment guide 30, where the handle 40 offers better grip of and control over the alignment guide 30 and therefore better positioning of the distractor/nosecone portion 10. The handle 40 may be made of any suitable material, including plastic, stainless steel, etc. The handle 40 may slide over the alignment guide 30, or be threaded onto the alignment guide 30, or attached in any other suitable fashion, as long as it is removable later to allow installation of the body portion 20. In one embodiment, the handle 40 has a bore, the alignment guide 30 slides into the bore, and a set screw or other fastening device fixes the handle 40 firmly to the alignment guide 30 for as long as the handle 40 is needed for placement of the distractor/nosecone portion 10.

When the distractor/nosecone portion 10 is in position, the handle 40 is removed from the alignment guide 30. With the distractor/nosecone portion 10 in place, appropriate disc height restoration can be confirmed. In addition, the length of the implant body portion 20 to be implanted may be measured by reference to markings on the alignment guide 30, and the body portion 20 can be cut to the appropriate length before implantation. The implant body portion 20 is then translated along the alignment guide 30 and brought into close contact with the distractor/nosecone portion 10. Fixation between the body portion 20 and the distractor/nosecone portion 10 may be through the use of Morse taper, mechanical interlock, etc. Moreover, the alignment guide 30 may be used for ancillary device implant portion fixation.

The section of the alignment guide 30 which remains exposed after coupling the distractor/nosecone portion 10 and the body portion 20 may be removed by cutting the alignment guide 30 or by bending/breaking the alignment guide 30 at pre-established notches. In embodiments in which the alignment guide 30 is not integral with the distractor/nosecone portion 10, the distractor/nosecone portion 10 may be withdrawn—either by simply pulling the alignment guide 30 out of the bore 14 if the bore 14 is smooth, or by unscrewing the alignment guide 30 from the bore 14 if the bore 14 is threaded.

With further reference to FIGS. 3-6, an embodiment of the disclosed device 2 is shown in connection with an ALIF procedure. The ALIF (anterior) procedure is performed from the front of the patient, and may be used to correct the patient's lateral-view spinal curvature. With reference to FIG. 3, an anterior view shows positioning of the distractor/nosecone portion 10, in a posterior interbody space between adjacent vertebrae 100 and 102. The alignment guide 30 is visible in FIG. 3, but the handle 40 is omitted for clarity. FIG. 4 is a lateral view of the same procedural step as in FIG. 3 which shows reestablishment and/or maintenance of the posterior interbody height and foraminal volume with the distractor/nosecone portion 10 in place. The alignment guide 30 extends from the distractor/nosecone portion 10, and the handle 40 is still in place on the alignment guide 30.

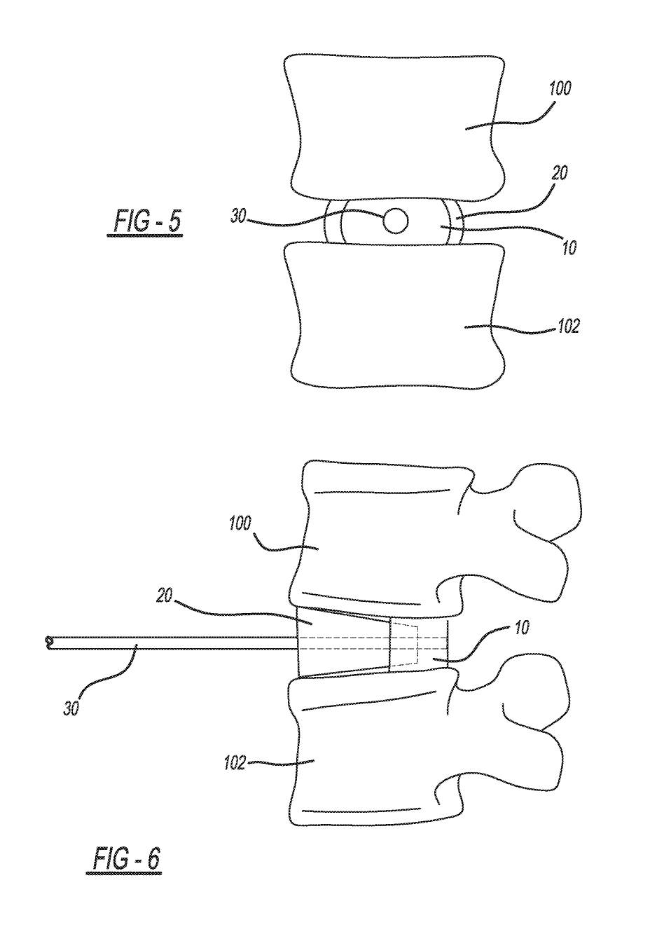

With reference to FIGS. 5 and 6, anterior and lateral views, respectively, of placement of the body portion 20 is shown. The body portion 20 of the device may be used to “dial in” lordosis (side-view spinal curvature) by adjusting the enforced angle between the vertebrae 100/102 while the posterior height is maintained. The body portion 20 of the device 2 may be wedge-shaped in order to facilitate establishment of the desired angle between the vertebrae 100/102 and achieve the desired lordosis. In another embodiment, the body portion 20 may be vertically expandable in order to facilitate establishment of the desired angle between the vertebrae 100/102 and achieve the desired lordosis.

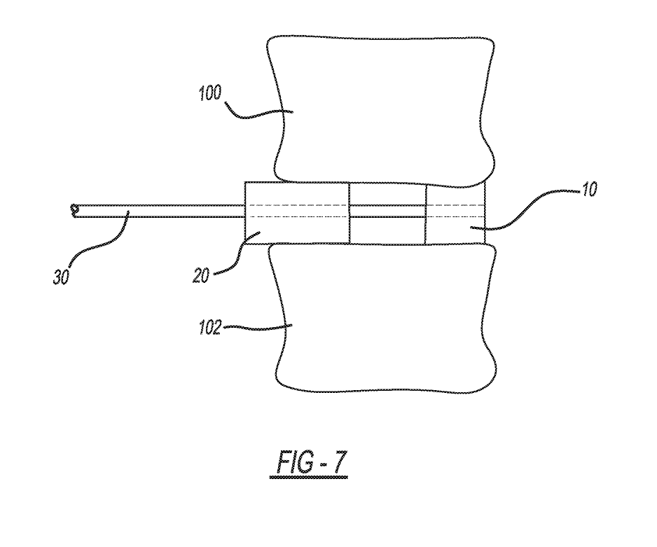

Now referring to FIG. 7, an embodiment of the disclosed device 2 is shown in connection with a LLIF procedure. The LLIF (lateral) procedure is performed from the side of the patient, and may be used to increase intervertebral space, to correct asymmetrical spinal curvature, or for spinal fusion. In an anterior (front) view, the distractor/nosecone portion 10 maintains the distal interbody height allowing for placement of the body portion 20 of the implant using the alignment guide 30 with markedly lessened risk of endplate damage as compared to conventional implants and procedures. The body portion 20 shown in FIG. 7 has constant thickness, as opposed to the wedge-shaped body portion 20 of FIG. 6.

As mentioned earlier, the alignment guide 30 may be fixed to the distractor/nosecone portion 10, or may be removable from the distractor/nosecone portion 10. Thus, in some embodiments, the alignment guide 30 is removed from the patient after the distractor/nosecone portion 10 and the body portion 20 have been placed, and in some embodiments, the alignment guide 30 remains inside the patient after the distractor/nosecone portion 10 and the body portion 20 have been placed. For embodiments where the alignment guide 30 remains inside the patient after the distractor/nosecone portion 10 and the body portion 20 have been placed between the vertebrae, it is advantageous to fix the body portion 20 to the alignment guide 30 in order to create a rigid assembly. Several different techniques are envisioned for fixing the body portion 20 to the alignment guide 30. These techniques include bending the alignment guide 30 over around the external end of the body portion 20, adding a locking collar over the alignment guide 30 up against the body portion 20, and any other mechanical means of attachment.

Although the devices and systems of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

What is claimed is:

1. A modular implant device for placement in a space between two adjacent vertebrae during a surgical procedure, said implant device comprising:
a distractor/nosecone portion configured for placement in a first location between the vertebrae, said distractor/nosecone portion providing distraction and fixation of a desired vertical separation between the vertebrae at the first location;
an alignment guide being a long slender element with a first end inserted into the distractor/nosecone portion and extending away from the distractor/nosecone portion in a direction of a surgeon, where the alignment guide is used by the surgeon to position the distractor/nosecone portion in the first location between the vertebrae; and
a body portion configured for placement in a second location between the vertebrae, said body portion providing distraction and fixation of a desired vertical separation between the vertebrae at the second location, where the body portion includes a bore to receive the alignment guide, and where the body portion is translated along the alignment guide into position between the vertebrae at the second location

2. The implant device of claim 1 wherein the surgical procedure is an ALIF (Anterior Lumbar Interbody Fusion), a PLIF (Posterior Lumbar Interbody Fusion), a TLIF (Transforaminal Lumbar Interbody Fusion) or an LLIF (Lateral Lumbar Interbody Fusion).

3. The implant device of claim 1 wherein the distractor/nosecone portion has a height which is adjustable after the implant device is in place between the two adjacent vertebrae.

4. The implant device of claim 1 further comprising a handle attachable to the alignment guide at a second end distal the distractor/nosecone portion, where the handle is configured to be attached to the alignment guide to aid in positioning the distractor/nosecone portion between the vertebrae, and the handle is removed from the alignment guide before placing the body portion of the implant device onto the alignment guide.

5. The implant device of claim 1 wherein the body portion has a wedge shape, where the vertical separation of the vertebrae at the second location increases as the body portion is inserted further into the space between the vertebrae.

6. The implant device of claim 1 wherein the body portion and the distractor/nosecone portion are configured with mating geometric features suitable for coupling the body portion and the distractor/nosecone portion together after the implant device is in place between the two adjacent vertebrae.

7. The implant device of claim 1 wherein the alignment guide and the bore in the body portion have a circular cross-section which allows rotation of the body portion relative to the distractor/nosecone portion when placing the body portion between the adjacent vertebrae during the surgical procedure.

8. The implant device of claim 1 wherein the alignment guide and the bore in the body portion have a non-circular cross-section which prevents rotation of the body portion relative to the distractor/nosecone portion when placing the body portion between the adjacent vertebrae during the surgical procedure.

9. The implant device of claim 1 wherein the alignment guide has measurement markings along its length suitable for measuring a required length of the body portion after the distractor/nosecone portion has been placed between the adjacent vertebrae, where the body portion can be cut to the required length before being placed between the adjacent vertebrae.

10. The implant device of claim 1 wherein the first end of the alignment guide is permanently fixed in the distractor/nosecone portion and the alignment guide remains inside a patient as part of the implant device after the surgical procedure.

11. The implant device of claim 10 wherein the alignment guide is configured to be fastenable to the body portion after the implant device is in place between the two adjacent vertebrae.

12. The implant device of claim 1 wherein the first end of the alignment guide is removable from the distractor/nosecone portion and the alignment guide is removed from the distractor/nosecone portion and the body portion after the body portion has been placed in the second location between the vertebrae.

13. An apparatus for use in intervertebral surgical procedures, said apparatus comprising:
a distractor/nosecone portion configured for placement in a first location between two adjacent vertebrae, said distractor/nosecone portion providing distraction and fixation of a desired vertical separation between the vertebrae at the first location;
an alignment guide being a long slender element with a first end permanently fixed in the distractor/nosecone portion and extending away from the distractor/nosecone portion in a direction of a surgeon, where the alignment guide is used by the surgeon to position the distractor/nosecone portion in the first location between the vertebrae;
a handle attachable to the alignment guide at a second end distal the distractor/nosecone portion, where the handle is configured to be attached to the alignment guide to aid in positioning the distractor/nosecone portion between the vertebrae, and the handle is removed from the alignment guide after positioning the distractor/nosecone portion between the vertebrae; and
a body portion configured for placement in a second location between the vertebrae, said body portion providing distraction and fixation of a desired vertical separation between the vertebrae at the second location, where the body portion includes a bore to receive the alignment guide, and where the body portion is translated along the alignment guide into position between the vertebrae at the second location.

14. The apparatus of claim 13 wherein the alignment guide is configured to be fastenable to the body portion after the body portion is in place between the two adjacent vertebrae.

15. The apparatus of claim 13 wherein the alignment guide has measurement markings along its length suitable for measuring a required length of the body portion after the distractor/nosecone portion has been placed between the adjacent vertebrae, where the body portion can be cut to the required length before being placed between the adjacent vertebrae.

16. The apparatus of claim 13 wherein the distractor/nosecone portion has a height which is adjustable after the distractor/nosecone portion is in place between the two adjacent vertebrae.

17. A method for placement of an implant device between two adjacent vertebrae during a surgical procedure, said method comprising:
placing a distractor/nosecone portion of the implant device in a desired nosecone position between the two adjacent vertebrae, where the distractor/nosecone portion establishes a vertical separation distance between the vertebrae at a first peripheral location on the vertebrae, and where the distractor/nosecone portion has an alignment guide protruding therefrom in a direction toward a surgeon, and the alignment guide is a long slender element configured to facilitate the placement of the distractor/nosecone portion at the desired nosecone position;
placing a body portion of the implant device onto the alignment guide by inserting an end of the alignment guide opposite the distractor/nosecone portion into a bore of the body portion; and
sliding the body portion along the alignment guide until the body portion reaches a desired body position between the two adjacent vertebrae, where the body portion establishes a vertical separation distance between the vertebrae at a second peripheral location on the vertebrae and thereby also establishes an angle between the vertebrae.

18. The method of claim 17 further comprising rigidly fixing the alignment guide to the body portion after the body has been placed in the desired body position between the vertebrae.

19. The method of claim 17 further comprising attaching a handle to the alignment guide before placing the distractor/nosecone portion in the desired nosecone position between the two adjacent vertebrae, and removing the handle from the alignment guide before placing the body portion of the implant device onto the alignment guide.

20. The method of claim 17 wherein the surgical procedure is an ALIF (Anterior Lumbar Interbody Fusion), a PLIF (Posterior Lumbar Interbody Fusion), a TLIF (Transforaminal Lumbar Interbody Fusion) or an LLIF (Lateral Lumbar Interbody Fusion).
